(19) United States
(12) Patent Application Publication
(10) Pub. No.: US 2009/0028906 A1
(43) Pub. Date: Jan. 29, 2009

(54) Process for providing a temperature-stable muscle relaxant on the basis of the neurotoxic component of botulinum toxin in a solid form

(75) Inventors: Swen Grein, Bad Vilbel (DE); Gerd J. Mander, Friedberg (DE); Matthias Marx, Mannheim (DE)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: MERZ PHARMA GMBH & CO. KGAA, Frankfurt Main (DE)

(21) Appl. No.: 12/154,983

(22) Filed: May 29, 2008

Related U.S. Application Data
(60) Provisional application No. 60/998,858, filed on Oct. 12, 2007.
(60) Provisional application No. 60/932,624, filed on Jun. 1, 2007.

(30) Foreign Application Priority Data
Jun. 1, 2007 (EP) 07010912.9
Oct. 12, 2007 (EP) 07020025.8

(51) Int. Cl. A61K 39/08; A61P 21/02

(52) U.S. Cl. 424/239.1

(57) ABSTRACT

The present invention relates to a method for providing a muscle relaxant at temperatures above 20° C., wherein said muscle relaxant is a solid dry composition comprising the neurotoxic component of botulinum toxin free of complexing proteins.

### FIELD OF THE INVENTION

The present invention provides a process for providing a muscle relaxant at elevated temperatures, wherein said muscle relaxant is a solid dry composition comprising the neurotoxic component of botulinum toxin free of complexing proteins.

### BACKGROUND OF THE INVENTION

Botulinum toxin is produced by the bacterium Clostridium. There are seven antigenically distinct serotypes of botulinum toxin, namely botulinum toxin A, B, C, D, E, F and G. Botulinum toxins are released from lysed Clostridium cultures generally in the form of a complex, i.e. the sub-unit responsible for the toxic properties of the Botulinum toxin (the so-called “neurotoxic component”), is associated with other bacterial proteins, which together form a toxin complex. The molecular weight of this complex may vary from about 300,000 to about 900,000 Da. The complexing proteins are, for example, various hemagglutinins. The proteins of this toxin complex are not toxic themselves but are believed to provide stability to the neurotoxic component and are responsible for oral toxicity in botulinum intoxications. Unlike the toxin complex, the neurotoxic component in its isolated and pure form, i.e. devoid of any complexing Clostridium proteins, is acid labile and does not resist the aggressive environment in the gastrointestinal tract.

The neurotoxic component of the botulinum toxin complex is initially formed as a single polypeptide chain, having in the case of serotype A a molecular weight of approximately 150 kDa. In other serotypes the neurotoxic component has been observed to vary between about 145 and about 170 kDa, depending on the bacterial source. In the case of serotype A, for example, proteolytic processing of the polypeptide results in an activated polypeptide in the form of a dichain polypeptide consisting of a heavy chain and a light chain, which are linked by a disulfide bond. In humans, the heavy chain mediates binding to pre-synaptic cholinergic nerve terminals and internalization of the toxin into the cell. The light chain is believed to be responsible for the toxic effects, acting as zink-endopeptidase and cleaving specific proteins responsible for membrane fusion (SNARE complex) (see e.g. Montecucco C., Shiavo G., Rosetto O: The mechanism of action of tetanus and botulinum neurotoxins. Arch Toxicol. 1996; 18 (Suppl.): 342-354)).

By disrupting the process of membrane fusion within the cells, botulinum toxins prevent the release of acetylcholine into the synaptic cleft. The overall effect of botulinum toxin at the neuro-muscular junction is to interrupt neuro-muscular transmission, and, in effect, denervate muscles. Botulinum toxin also has activity at other peripheral cholinergic synapses, causing a reduction of salivation or sweating.

The neurotoxic subunit of the Botulinum toxin complex is referred herein as the “neurotoxic component” or the “neurotoxic component free of complexing proteins”.

The terms “botulinum toxin” or “botulinum toxins” as used throughout the present applicaton, refer to the neurotoxic component devoid of any other clostridial proteins, but also to the “botulinum toxin complex”: The term “botulinum toxin” is used herein in cases when no discrimination between the complex or the neurotoxic component is necessary or desired. The complex usually contains additional, so-called “non-toxic” proteins, which we will refer to as “complexing proteins” or “bacterial proteins”.

“BoNT” or “NT” are common used abbreviations relating to the NT compound of botulinum toxin free of complexing proteins.

Despite its toxic effects, botulinum toxin complex has been used as a therapeutic agent in a large number of diseases. Botulinum toxin serotype A was approved for human use in the United States in 1989 for the treatment of strabism, blepharospasm, and other disorders. It is commercially available as Botulinum toxin A protein complex, for example, under the tradename BOTOX (Allergan Inc) or under the tradename DYSPORT (Ipsen Ltd). For therapeutic application the complex is injected directly into the muscle to be treated. At physiological pH, the toxin is released from the protein complex and the desired pharmacological effect takes place.

Before administering it to a patient, typically intramuscular directly into the affected muscle, said composition is dissolved in physiological saline solution.

With regard to the composition and dosing of the medicament on the basis of botulinum toxin, and in regard to the composition, dosing and frequency of administration of the medicament on the basis of the neurotoxic component of botulinum toxin, reference is made to U.S. 60/817,756.

In addition to the above-recited function of the complexing proteins it has been speculated that they also protect the neurotoxic component of the Botulinum toxin complex from harsh environmental conditions, and that the neurotxic component as such is highly susceptible to degradation and/or inactivation, especially when subjected to short-term temperature stress, such as storage and/or transport in warm to hot climate or during summer in general.

For said reason, utmost care is generally taken in the past to prevent the medicaments on the basis of the Botulinum toxins and those on the basis of the neurotoxic component of Botulinum toxin in particular, from reaching a temperature of above 4° C., e.g. close to 20° C. In most cases the vials containing the solid dry composition comprising the botulinum toxins were stored frozen (around −20° C.), under ice or at least in a refrigerator (around 4° C.). The necessary cooling means added to the cost of providing the medicaments.

In view of the above situation, the applicants undertook studies regarding the temperature stability of muscle relaxants on the basis of botulinum toxin. It was surprisingly found that the neurotoxic component of botulinum toxin is significantly more temperature stable than expected in the art.

### SUMMARY OF THE INVENTION

The present invention relates to a process for providing a muscle relaxant at elevated temperatures above 20° C., e.g. above 30, or above 45 wherein said muscle relaxant is a solid dry composition comprising the neurotoxic component of botulinum toxin free of complexing proteins, preferably said process involves storage and/or transport. In another embodiment, said provision is a step within a process for preparing said muscle relaxant, e.g. a step carried out after the proteins including the neurotoxic component of botulinum toxin have been dried.

In one embodiment a muscle relaxant at temperatures above 45° C. is provided, wherein said muscle relaxant is a solid dry composition comprising the neurotoxic component of botulinum toxin free of complexing proteins.

In another embodiment said provision involves storage and/or transport and/or is a step within a process for preparing said muscle relaxant.

In another embodiment the muscle relaxant is subjected to a temperature above 45° C. and up to 70° C. for a time period not exceeding 90 days. In a further embodiment the time period ranges from 10 minutes to 90 days. In yet a further embodiment the temperature is between 45 and 60° C. and the time period ranges from 10 minutes to 90 days. In yet a further embodiment the time period ranges from 10 minutes to 30 days. In yet a further embodiment the temperature is between 65° C. and 70° C. and the time period ranges from 10 minutes to 10 days.

In another embodiment said muscle relaxant is transported and/or stored without any device for artificial cooling.

In another embodiment the composition is a lyophilysate of the neurotoxic component of botulinum toxin.

In another embodiment the composition further comprises sucrose and/or human serum albumin.

In another embodiment the composition further comprises at least one component selected from the group consisting of a cryoprotectant, a stabilizer, a pH buffer, an excipient, different from sucrose and human serum albumin, respectively, and mixtures thereof.

In another embodiment the neurotoxic component is the neurotoxic component of Botulinum toxin type A.

### BRIEF DESCRIPTION OF THE DRAWINGS

FIG. 1 shows the impact of temperature on the biological activity of Xeomin® after storage at 60° C. for a period of up to 30 days; the biological activity was tested at the indicated points in time;

FIG. 2 shows the impact of temperature on the biological activity of Xeomin® after storage at 70° C. for a period of up to 10 days; the biological activity was tested at the indicated points in time;

FIG. 3 shows the impact of temperature on the biological activity of Xeomin® after storage at 80° C. for a period of up to 10 days; the biological activity was tested at the indicated points in time.

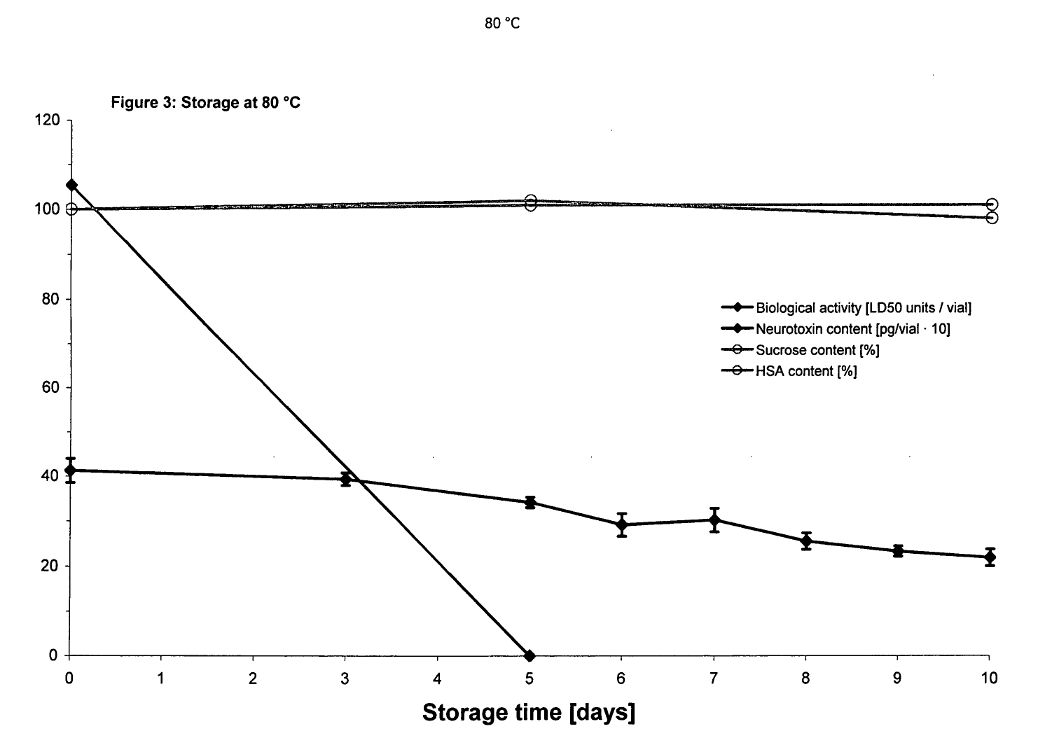

### DETAILED DESCRIPTION OF THE INVENTION

The present invention relates to a process for providing a muscle relaxant at temperatures above 20° C., wherein said muscle relaxant is a solid dry composition comprising the neurotoxic component of botulinum toxin free of complexing agents. In another embodiment said muscle relaxant is provided at temperatures of 45° C. and/or above, wherein said muscle relaxant is a solid dry composition comprising the neurotoxic component of botulinum toxin free of complexing agents. Within this invention, the term “providing” includes any kind of provision of the muscle relaxant defined herein, in particular storage, transport, or a step within the preparation of said muscle relaxant. The term “providing” also includes steps wherein the muscle relaxant is subjected to a rise in temperature thereof from the frozen (−20° C.), or cooled (+4° C.) state to a temperature of above 20° C. In another embodiment said muscle relaxant is provided at temperatures of 45° C. and/or above, wherein said muscle relaxant is a solid dry composition comprising the neurotoxic component of botulinum toxin free of complexing agents.

Within this invention, all forms of the neurotoxic component of botulinum toxin, in particular the various serotypes are to be used. In addition thereto, modified and/or recombinantly produced neurotoxic components of botulinum toxins including the respective mutations, deletions, etc. are also within the scope of the present invention. With respect to suitable mutants, reference is made to WO 2006/027207 A1, WO 2006/114308 A1 and EP07014785.5, which are fully incorporated by reference herein. Furthermore, within the present invention, mixtures of various serotypes (in the form the neurotoxic component and/or recombinant forms thereof, e.g. mixtures of botulinum neurotoxins of types A and B) may be used. The present invention, however, also refers to neurotoxins which are chemically modified, e.g. by pegylation, glycosylation, sulfatation, phosphorylation or any other modification, in particular of one or more surface or solvent exposed amino acid(s).

In one embodiment said composition comprises the neurotoxic component of Botulinum toxin type A. In another embodiment the botulinum toxin is botulinum toxin of the antigenically distinct serotypes A, B, C, D, E, F, or G. Wherever the botulinum toxin serotype A, B, C, D, E, F or G are mentioned, also known variants of the serotypes are encompassed, like serotypes A1, A2, A3, B1, B2, B3, C1, C2, C3, D1, D2, D3, E1, E2, E3, F1, F2, F3, or G1, G2, G3. According to the present invention one serotype or a mixture of different serotypes maybe used. In one embodiment the botulinum toxin is botulinum toxin A.

In another embodiment, also isoforms, homologs, orthologs and paralogs of Botulinum toxin are encompassed, which show at least 50%, at least 60%, at least 70%, at least 80%, at least 90% or up to 100% sequence identity. The sequence identity can be calculated by any algorithm suitable to yield reliable results, for example by using the FASTA algorithm (W. R. Pearson & D. J. Lipman PNAS (1988) 85:2444-2448).

The term “neurotoxic component” also includes functional homologs found in the other serotypes of Clostridium botulinum. In one embodiment of the present invention, the neurotoxic component is devoid of any other C. botulinum protein, in one embodiment also devoid of RNA, which might potentially be associated with the neurotoxic component. The neurotoxic component may be the single chain precursor protein of approximately 150 kDa or the proteolytically processed neurotoxic component, comprising the light chain (L_{c}) of approximately 50 kDa and the heavy chain (H_{c}) of approximately 100 kDa, which may be linked by one or more disulfide bonds (for a review see e.g. Simpson L L, Ann Rev Pharmacol Toxicol. 2004; 44:167-93). In another embodiment a mixture of single chain and processed neurotoxic components maybe used.

In accordance with the teaching of the present invention it is possible that the medicament contains no proteins found in the botulinum toxin complex other than the neurotoxic component. The precursor of the neurotoxic component may be cleaved or uncleaved, however, in one embodiment the precursor has been cleaved into the heavy and the light chain. As pointed out elsewhere herein, the polypeptides may be of wild-type sequence or may be modified at one or more residues. Modification comprises chemical modification e.g. by glycosylation, acetylation, acylation, amidation or the like, which may be beneficial e.g. to the uptake or stability of the polypeptide. The polypeptide chain of the neurotoxic component may, however, alternatively or additionally be modified by addition, substitution or deletion of one or more amino acid residues.

The neurotoxic component referred to herein above, may be part of a composition or a pharmaceutical composition. This pharmaceutical composition to be used herein may comprise botulinum toxin, e.g. in the form of neurotoxic component as the sole active component or may contain additional pharmaceutically active components e.g. analgesic and/or further neurotoxins.

A “pharmaceutical composition” as used herein is a formulation in which an active ingredient for use as a medicament or a diagnostic is contained or comprised. Such pharmaceutical composition may be suitable for diagnostic or therapeutic administration (i.e. by intramuscular or subcutaneous injection) to a human patient.

In one embodiment of the present invention, the composition may comprise the neurotoxic component and a hyaluronic acid and/or a polyvinylpyrrolidone and/or a polyethleneglycol, such composition being optionally pH stabilized by a suitable pH buffer, in particular by a sodium acetate buffer and/or a cryoprotectant polyalcohol.

In one embodiment, the neurotoxic component has a biological activity of 50 to 250 LD₅₀ units per ng neurotoxic component, as determined in a mouse LD₅₀ assay. In another embodiment, the neurotoxic component has a biological activity of about 150 LD₅₀ units. Said 150 LD₅₀ units refer to units per nanogram. Generally, the pharmaceutical composition of the present invention comprises neurotoxic component in a quantity of about 6 pg to about 30 ng.

A pharmaceutical composition comprising the neurotoxic component of botulinum toxin type A in isolated form is commercially available in Germany from Merz Pharmaceuticals GmbH under the trademark Xeomin®. The production of the neurotoxic component of botulinum toxin type A and B are described, for example, in the international patent applications WO 00/74703 and WO 2006/133818.

In one embodiment, said composition comprises the neurotoxic component of botulinum toxin type A. Said composition is a solid dry composition of the neurotoxic component of botulinum toxin. In another embodiment the composition further comprises e.g. sucrose or human serum albumin or both, still another embodiment the ratio of human serum albumin to sucrose is about 1:5. In one embodiment, the composition is Xeomin®. In another embodiment, said human serum albumin is recombinant human serum albumin. Alternatively, said composition is free of mammalian derived proteins such as human serum albumin. Any such solution may provide sufficient neurotoxin stability by replacing serum albumin with other non-proteinaceous stabilizers (infra).

Within the present patent application, the use of a medicament based on the neurotoxic component of botulinum toxin type A, in another embodiment the product distributed by Merz Pharmaceutical under the trademark Xeomin® can be used. This is because the tendency of generating antibodies within the patient was found to be lower when applying pharmaceutical compositions on the basis of the neurotoxic component of botulinum toxin, such as Xeomin® compared to administering medicaments on the basis of the botulinum toxin type A complex. Without being bound to any theory, it is believed that the hemagglutinins within the botulinum toxin complex have an activating capability on the immune system.

With regard to the composition and dosing of the medicament on the basis of botulinum toxin, and in regard to the composition, dosing and frequency of administration of the medicament on the basis of the neurotoxic component of botulinum toxin, reference is made to PCT/EP2007/005754.

In one embodiment said composition is a lyophilisate of the neurotoxic component of botulinum toxin, said composition may further comprise sucrose and/or human serum albumin. In said latter composition the ratio of human serum albumin to sucrose maybe for example about 1:5. In one embodiment, the composition is Xeomin®.

Such composition may comprise additional excipients. The term “excipient” refers to a substance present in a pharmaceutical composition other than the active pharmaceutical ingredient present in the pharmaceutical composition. An excipient can be a buffer, carrier, antiadherent, analgesic, binder, disintegrant, filler, diluent, preservative, vehicle, cyclodextrin and/or bulking agent such as albumin, gelatin, collagen, sodium chloride, preservative, cryoprotectant and/or stabilizer.

A “pH buffer” refers to a chemical substance being capable to adjust the pH value of a composition, solution and the like to a certain value or to a certain pH range. In one embodiment this pH range can be between pH 5 to pH 8, in another embodiment pH 7 to pH 8, in yet another embodiment 7.2 to 7.6, and in yet a further embodiment a pH of 7.4. In another embodiment the pharmaceutical composition has a pH of between about 4 and 7.5 when reconstituted or upon injection, in yet another embodiment about pH 6.8 and pH 7.6 and in a further embodiment between pH 7.4 and pH 7.6.

In one embodiment the composition also contains a 1-100 mM sodium acetate buffer, in another embodiment 10 mM sodium acetate buffer.

The pH ranges indicated are only typical examples and the actual pH may include any interval between the numerical values given above. Suitable buffers which are in accordance with the teaching of the present invention are e.g. sodium-phosphate buffer, sodium-acetate buffer, TRIS buffer or any buffer, which is suitable to buffer within the above pH-ranges.

The term “room temperature” in this document refers to any temperature between +20° C. to +25° C., even more preferably any of the temperatures of +20° C., +21° C., +22° C., +23° C., +24° C. or +25° C. and any value in between.

“Stabilizing”, “stabilizes” or “stabilization” means that the active ingredient, i.e., the neurotoxic component in a reconstituted or aqueous solution pharmaceutical composition has greater than about 20%, 30%, 40%, 50%, 60%, 70%, 80%, 90%, and up to about 100% of the toxicity that the biologically active neurotoxic component had prior to being incorporated into the pharmaceutical composition.

Examples of such stabilizers are gelatin or albumin, in one embodiment of human origin or obtained from a recombinant source. Proteins from non-human or non-animal sources are also included. The stabilizers may be modified by chemical means or by recombinant genetics. In one embodiment of the present invention, it is envisaged to use alcohols, e.g., inositol, mannitol, as cryoprotectant excipients to stabilize proteins during lyophilization.

In another embodiment of the present invention, the stabilizer may be a non proteinaceous stabilizing agent comprising a hyaluronic acid or a polyvinylpyrrolidone, polyethylene glycol or any combination thereof. In another embodiment said polyvinylpyrrolidone maybe for example (Kollidon®). Further stabilizers maybe hydroxyethyl starch or alginate. Such compositions maybe optionally pH stabilized by a suitable pH buffer, in particular by a sodium acetate buffer, or a cryoprotectant or both. Said composition may comprise in addition to the mentioned stabilizers water and at least one polyalcohol, such as mannitol or sorbitol or mixtures thereof. It may also comprise mono-, di- or higher polysaccharides, such as glucose, sucrose or fructose. Such composition is considered to be a safer composition possessing remarkable stability.

The hyaluronic acid in the instant pharmaceutical composition is in one embodiment combined with the instant neurotoxic component in a quantity of 0.1 to 10 mg, especially 1 mg hyaluronic acid per ml in a 200 U/ml botulinum toxin solution.

The polyvinylpyrrolidone when present in the instant composition, is combined with the instant neurotoxic component in such a quantity to provide a reconstituted solution comprising 10 to 500 mg, especially 100 mg polyvinylpyrrolidone per ml in a 200 U/ml neurotoxic component of botulinum toxin solution. In another embodiment reconstitution is carried out in up to 8 ml solution. This results in concentrations of down to 12.5 mg polyvinylpyrrolidone per ml in a 25 U/ml neurotoxic component solution. In another embodiment, the subject solution also contains a 1-100 mM, in yet another embodiment 10 mM sodium acetate buffer. The polyethyleneglycol in the instant pharmaceutical composition is in one embodiment combined with the instant neurotoxic component in a quantity to provide a reconstituted solution comprising 10 to 500 mg, in yet another embodiment 100 mg polyethyleneglycol per ml in a 200 U/ml botulinum toxin solution. In another embodiment, the subject solution also contains a 1-100 mM, in yet another embodiment 10 mM sodium acetate buffer.

The pharmaceutical composition in accordance with the present invention in one embodiment retains its potency substantially unchanged for six month, one year, two year, three year and/or four year periods when stored at a temperature between about +80° C. and about −20° C. Additionally, the indicated pharmaceutical compositions may have a potency or percent recovery of between about 20% and about 100% upon reconstitution.

In another embodiment the stabilizers are selected from the stabilizers known in the art which are not thioalkyls, methionin or trehalose.

“Cryoprotectant” refers to excipients which result in a neurotoxic component in a reconstituted or aqueous solution pharmaceutical composition that has greater than about 20%, 30%, 40%, 50%, 60%, 70%, 80%, 90%, and up to about 100% of the toxicity that the biologically active neurotoxic component had prior to being freeze-dried in the pharmaceutical composition. Said “Cryoprotectant” refers to excipients which result in an active ingredient.

In another embodiment, the composition may contain a polyalcohol as cryoprotectant. In one embodiment the composition my contain a polyhydroxy compound. Examples of polyalcohols that might be used include, e.g., inositol, mannitol and other non-reducing alcohols. Some embodiments of the composition do not comprise a proteinaceous stabilizer, or do not contain trehalose or maltotriose or lactose or sucrose or related sugar or carbohydrate compounds which are sometimes used as cryoprotectants.

The terms “preservative” and “preservatives” refer to a substance or a group of substances, respectively, which prevent the growth or survival of microorganisms, insects, bacteria or other contaminating organisms within said composition. Preservatives also prevent said composition from undesired chemical changes. Preservatives which can be used in the scope of this patent are all preservatives of the state of the art known to the skilled person. Examples of preservatives that might be used include, inter alia, e.g. benzylic alcohol, benzoic acid, benzalkonium chloride, calcium propionate, sodium nitrate, sodium nitrite, sulphites (sulfur dioxide, sodium bisulfite, potassium hydrogen sulfite, etc.), disodium EDTA, formaldehyde, glutaraldehyde, diatomaceous earth, ethanol, methyl chloroisothiazolinone, butylated hydroxyanisole and/or butylated hydroxytoluene.

The term “analgesic” relates to analgesic drugs that act in various ways on the peripheral and central nervous systems and includes inter alia Paracetamol® (acetaminophen), the nonsteroidal anti-inflammatory drugs (NSAIDs) such as the salicylates, narcotic drugs such as morphine, synthetic drugs with narcotic properties such as Tramadol®, and various others. Also included is any compound with a local analgesic effect such as e.g. lidocaine, benzylic alcohol, benzoic acid and others.

In one embodiment the analgesic is part of the composition, in another embodiment, the analgesic is administered before, during or after the treatment with the chemodenervating agent.

Typically, the above referenced provision of the muscle relaxant involves storage and/or transport of the same, or is a step within a process for preparing said muscle relaxant, more preferably a step carried out after the proteins including the neurotoxic component of botulinum toxin have been dried, at elevated temperatures, respectively. By “elevated temperatures” temperatures above 20° C., e.g. above 25° C., or above 30° C. are meant. In exceptional cases, i.e. in an environment where the muscle relaxant on the basis of the neurotoxic component of botulinum toxin is stored below 0° C., the term “elevated temperatures” refers to temperatures above 0° C., or above 4° C., or above 10° C., above 20, or or 30° C., respectively.

In another embodiment, the muscle relaxant is subjected to a temperature lying in the range of above 30° C. and up to 70° C. for a time period not exceeding 90 days. As the person skilled in the art is perfectly aware of, the time period for which the muscle relaxant is subjected to the respective temperature can be any time interval between a few minutes and 90 days. Typically, taking into account the circumstances of providing such a muscle relaxant, and in particular the situation where storing and/or transportation is involved the time period will not be less than 10 minutes. These short periods are particularly important under circumstances, wherein after transportation and before storage in a hot climate, the muscle relaxant is subjected to direct sunlight, e.g. on an airport or on the street. Typical time periods within the present invention are therefore, 10 minutes, 30 minutes, 1 hour, 3 hours, 4 hours, 5 hours, 6 hours, 7 hours, 8 hours, 1 days, 2 days, 3 days, 4 days, 5 days, 6 days, 7 days, 8 days, 9 days, 10 days, 2 weeks, 3 weeks, 1 month, 2 months, 3 months (90 days). Needless to say, that the time periods mentioned above are only typical examples and the actual time periods may be longer or shorter and include any interval between the numerical values given above.

As to the temperature, to which the muscle relaxant is subjected, typically a lower limit of a temperature of above 20° C. is envisioned by the person skilled in the art. With respect to the temperatures and temperature ranges specified herein, the person skilled in the art understands that the upper temperature to which the muscle relaxant/composition is subjected is preferably not above 70° C. This is, the temperatures to which the muscle relaxant is subjected preferably lie in a range of above 20° C. and up to 70° C. Therefore, within the present invention, the muscle relaxant is subjected to a temperature above 20° C., or above 25° C., or above 30° C., or above 35° C., or above 40° C., or above 45° C., or above 50° C., or above 60° C., or above 65° C., to up to 70° C., respectively. Again, any specific temperature between the given values of above 20° C. and up to 70° C. as well as respective temperature intervals, which may be the result of the environment at which the muscle relaxant is provided, preferably transported and/or stored, lies within the present invention.

The following temperature and time intervals represent embodiments of the present invention. According to a first embodiment the muscle relaxant is subjected to a temperature above 30° C. and up to 70° C. for a time period not exceeding 90 days, more preferably to a temperature above 30° C. and up to 70° C. for a time period ranging from 10 minutes to 90 days, more preferably at a temperature of between 40 and 60° C. and a time period ranging from 10 minutes to 90 days.

In a further embodiment the time period ranges from 10 minutes to 30 days, while the temperature ranges from above 30° C. to up to 70° C., or from 40° C. to 60° C., or from 50° C. to 60° C.

In a further embodiment, representing extreme conditions, the temperature lies in the range of between 65° C. and 70° C. and the time period for which the muscle relaxant is subjected to said temperature lies in the range of from 10 minutes to 10 days, or from 10 minutes to 3 days.

Due to the findings on which the present invention is based, it is now possible to provide a muscle relaxant as outlined above without using a device for artificial cooling. This finding is particularly important for the transportation and/or storage of such a muscle relaxant. Furthermore, the invention is particularly relevant in an environment of elevated temperature, possibly together with an increased humidity.

The present invention is now further exemplified by way of the non-limited examples recited hereinunder.

### EXAMPLES

The examples have been conducted with the commercially available product Xeomin®. Xeomin® is a lyophilized powder containing botulinum neurotoxin type A (150 kD) as active ingredient. The toxin is present in nicked double chain form, i.e. it contains a heavy and a light chain. The toxin is obtained from Clostridium botulinum cultures (strain ATCC 3205). It has been purified to such a degree that it is free of any complexing proteins. Xeomin® further comprises human serum albumin and sucrose.

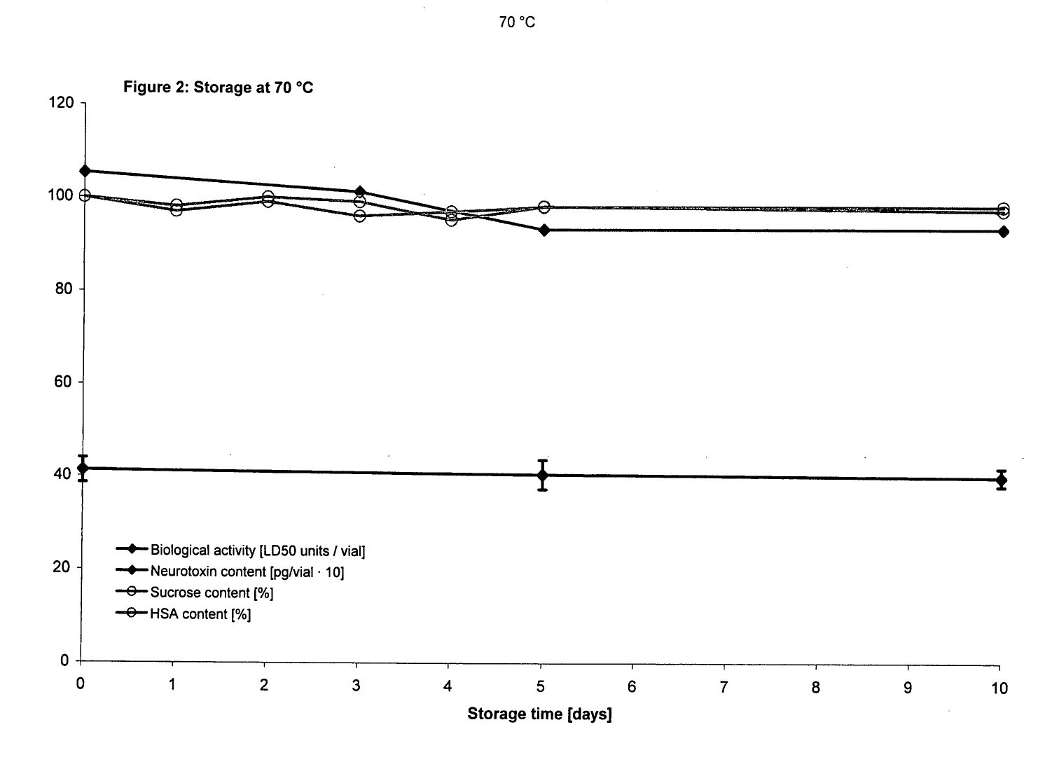

Samples of Xeomin® (unopened vials not reconstituted) were stored at temperatures of 60° C. for 30 days (example 1), at 70° C. for 10 days (FIG. 2) and 80° C. for 10 days (comparative example 3), respectively. The storage was conducted using qualified incubators with narrow temperature tolerance (±2° C.). The samples were consecutively removed from the incubators in daily intervals and stored at 5° C. until analysis.

For evaluating the stability of Xeomin®, the biological activity by using the well-known mouse LD₅₀ assay, the content of the neurotoxic component in pg/vial×10, the sucrose content (in %) and the HSA content (in %) were determined, respectively. The above-mentioned methods were carried out in accordance with the requirements laid down in the European Pharmacopeia.

### Example 1

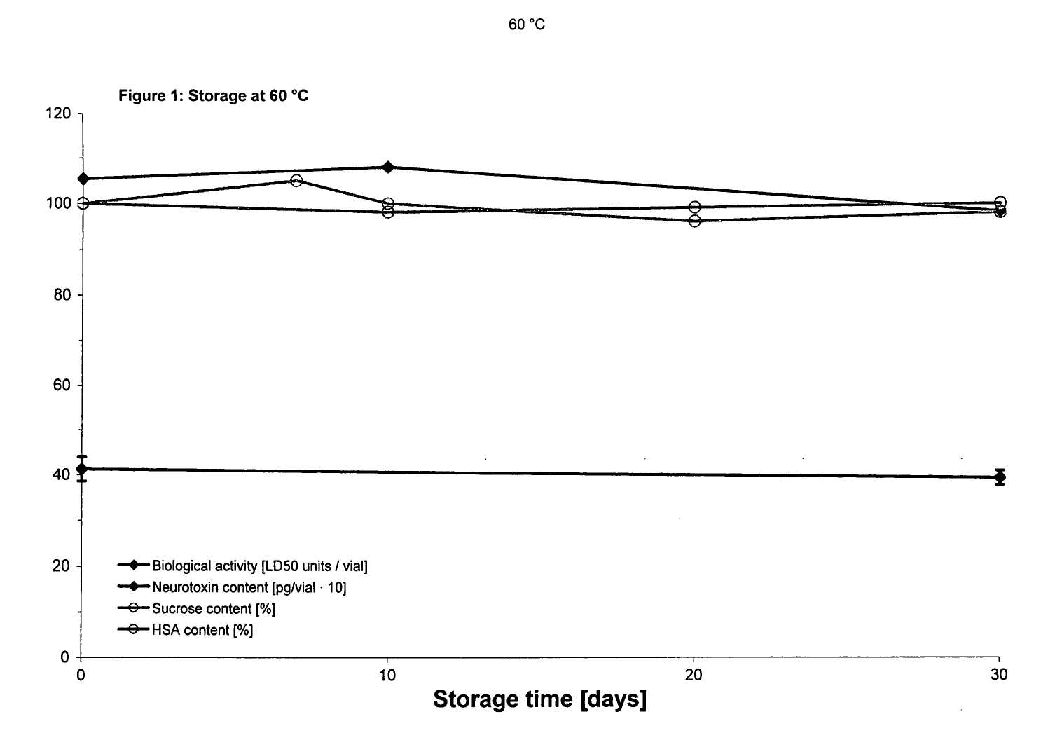

According to this example, Xeomin® was stored for up to 1 month at a temperature of 60° C. The results are shown in FIG. 1.

As it becomes apparent from said figure, the quality of Xeomin® is not affected by storage at 60° C. over a period of up to 1 month. Both the biological activity (LD50 assay) and the neurotoxin concentration (ELISA) remain virtually unchanged over the complete time of storage. In addition, the levels of human serum albumin (HSA) and sucrose show no significant variation over time. Taken together, all parameters of the shelf-life specification for Xeomin® are fulfilled after 1 month of storage at 60° C. Similarly, all stability data from storage conditions below 60° C. show no detrimental effect on the quality of the product (data not shown).

### Example 2

According to said example, Xeomin® is stored at a temperature of 70° C. for a period of up to 10 days. The results are shown in FIG. 2.

The quality of Xeomin®® is not significantly affected by storage at 70° C. over a period of up to 10 days. Again, the biological activity, the neurotoxin concentration as well as the HSA and sucrose content show no significant variation over time. Taken together, the example demonstrates that the quality of Xeomin® is not significantly affected by the storage at 70° C. for up to 10 days.

### Example 3 (Comparative)

In this (comparative) example, Xeomin® is subjected to storage at 80° C. for a period of up to 10 days. In contrast to the stability data at 60° C. and 70° C., a rapid decrease of the biological activity (LD₅₀ assay) can be observed. The neurotoxin is completely inactivated within 5 days, with a further reduction of activity during the first 3 days of storage. This example shows that at least temperatures of 80° C. have a detrimental effect on the stability of a muscle relaxant on the basis of the neurotoxic component of botulinum toxin already after a relatively short period of time.

1. A method for providing a muscle relaxant at temperatures above 20° C., wherein said muscle relaxant is a solid dry composition comprising the neurotoxic component of botulinum toxin free of complexing proteins.

2. The method of claim 1, wherein the method for providing involves storage and/or transport and/or is a step within a process for preparing said muscle relaxant.

3. The method of claim 1, wherein the muscle relaxant is subjected to a temperature above 20° C. and up to 70° C. for a time period not exceeding 90 days.

4. The method of claim 3, wherein the time period ranges from 10 minutes to 90 days.

5. The method of claim 3, wherein the temperature is between 20 and 60° C. and the time period ranges from 10 minutes to 90 days.

6. The method of claim 4, wherein the time period ranges from 10 minutes to 30 days.

7. The method of claim 3, wherein the temperature is between 20° C. and 70° C. and the time period ranges from 10 minutes to 10 days.

8. The method of claim 2, wherein said muscle relaxant is transported and/or stored without any device for artificial cooling.

9. The method of claim 1, wherein the composition is a lyophilysate of the neurotoxic component of Botulinum toxin

10. The method of claim 1, wherein the composition further comprises sucrose and/or human serum albumin.

11. The method of claim 1, wherein the composition further comprises at least one component selected from the group consisting of a cryoprotectant, a stabilizer, a pH buffer, an excipient, and mixtures thereof, wherein such component is not sucrose or human serum albumen.

12. The method of claim 1, wherein the neurotoxic component is the neurotoxic component of Botulinum toxin type A.
